(19) United States
(12) Patent Application Publication
(10) Pub. No.: US 2017/0058310 A1
(43) Pub. Date: Mar. 2, 2017

(54) BIOSENSING DEVICES AND METHODS OF USING AND PREPARING THE SAME

(71) Applicant: EMPIRE TECHNOLOGY DEVELOPMENT LLC, Wilmington, DE (US)

(72) Inventor: Vincenzo CASASANTA, III, Woodinville, WA (US)

(73) Assignee: EMPIRE TECHNOLOGY DEVELOPMENT LLC, Wilmington, DE (US)

(21) Appl. No.: 15/120,446

(22) PCT Filed: Feb. 19, 2014
(86) PCT No.: PCT/US14/17028

§ 371 Date: Aug. 19, 2016

(51) Int. Cl. C12Q 1/02; G01N 21/552

(52) U.S. Cl. CPC C12Q 1/02; G01N 21/554; G01N 2201/0683; G01N 2201/0636; G01N 2201/0628

(57) ABSTRACT

A biosensing device, as well as methods of forming a biosensing device and detecting presence of a biofilm are disclosed. The biosensing device may include a substrate, at least one radiation source on the substrate, at least one radiation detector on the substrate, and at least one reflector arranged on the substrate such that radiation emitted from the at least one radiation source is reflected toward the at least one radiation detector. The at least one radiation detector may be configured to detect an intensity of the radiation reflected from the at least one reflector. A biofilm growth on a portion of the at least one reflector may cause a change in the intensity of the radiation reflected from the at least one reflector relative to radiation reflected from the reflector in the absence of the biofilm growth.

### BACKGROUND

Growth of bacterial biofilms involves surface attachment of extracellular pili and excretion of proteins, polysaccharides, and nucleic acids. Some of these excreted items may be molecularly anchored to a surface of a host substrate.

Current methods of detecting attachment of the molecularly anchored items to the surface of the host substrate include various surface plasmon resonance (SPR) technologies. Most SPR systems are bench-top component ensembles that include traditional optical trains and detectors. Such systems are large, bulky, costly to build, costly to maintain, and costly to use. In addition, the systems are not easily transported or implemented, particularly in small scale applications such as medical devices and microelectromechanical systems (MEMS).

### SUMMARY

In an embodiment, a biosensing device may include a substrate, at least one radiation source on the substrate, at least one radiation detector on the substrate, and at least one reflector arranged on the substrate such that radiation emitted from the at least one radiation source is reflected toward the at least one radiation detector. The at least one radiation detector may be configured to detect an intensity of the radiation reflected from the at least one reflector. A biofilm growth on a portion of the at least one reflector may cause a change in the intensity of the radiation reflected from the at least one reflector relative to radiation reflected from the reflector in the absence of the biofilm growth.

In an embodiment, a method of forming a biosensing device may include providing a substrate, forming at least one radiation source on the substrate, forming at least one radiation detector on the substrate, and forming at least one reflector on the substrate. The at least one reflector may be configured to reflect radiation emitted from the at least one radiation source toward the at least one radiation detector. The at least one radiation detector may be configured to detect an intensity of the radiation reflected from the at least one reflector.

In an embodiment, a method of detecting a presence of a biofilm on a surface may include providing a biosensing device that includes a substrate, at least one radiation source on the substrate, at least one radiation detector on the substrate, at least one reflector arranged on the substrate such that radiation emitted from the at least one radiation source is reflected toward the at least one radiation detector, and a film of sensor material on a surface of the at least one reflector opposite to a surface where the radiation is reflected. The at least one radiation detector may be configured to detect an intensity of the radiation reflected from the at least one reflector. The method may also include emitting radiation from the radiation source toward the reflector at an angle and reflecting the radiation via the reflector toward the radiation detector. The radiation reflected from the reflector may be a specular reflection of the radiation incident on the reflector. The method may also include measuring an intensity of the radiation reflected from the reflector using the radiation detector, recording a baseline intensity value based on the intensity of reflected radiation from radiation incident on the reflector at the angle, and detecting a biofilm presence when a growth of the biofilm on the sensor material causes a change in the intensity of reflected radiation from radiation incident on the reflector at the angle with respect to the recorded baseline intensity value.

### BRIEF DESCRIPTION OF THE DRAWINGS

FIG. 1 depicts a cutaway side view of an illustrative biosensing device according to an embodiment.

FIG. 2 depicts a cutaway side view of an illustrative array of biosensing devices according to an embodiment.

FIG. 3 depicts a top view of an illustrative biosensing device according to an embodiment.

FIG. 4 depicts a detailed side view of an illustrative radiation source according to an embodiment.

FIG. 5 depicts a graphical view of an illustrative biofilm growth on a substrate according to an embodiment.

FIG. 6A depicts a graphical view of an illustrative interdigitated well structure according to an embodiment.

FIG. 6B depicts a graph of responsitivity of an illustrative photodetector to various wavelengths of light according to an embodiment.

FIG. 7A depicts a graph of a reflected intensity based on a specular reflectance angle of an illustrative surface with no bound molecules according to an embodiment.

FIG. 7B depicts a graph of a reflected intensity based on a specular reflectance angle of an illustrative surface with bound molecules according to an embodiment.

FIG. 8 depicts a flow diagram of an illustrative method of forming a biosensing device according to an embodiment.

FIG. 9 depicts a flow diagram of an illustrative method of forming a radiation source according to a first embodiment.

FIG. 10 depicts a flow diagram of an illustrative method of forming radiation source according to a second embodiment.

FIG. 11 depicts a flow diagram of an illustrative method of detecting a biofilm growth on a surface according to an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The present disclosure relates generally to a device that uses surface plasmon resonance to detect biofilm growth on a surface. In an embodiment, the device may detect biofilm growth on any surface, including surfaces that offer a site for free-floating microorganisms to colonize and multiply. One such illustrative surface may be a surface of a medical device, including an indwelling medical device. Illustrative medical devices may include, but are not limited to, catheters, cannulae, central lines, arterial lines, and/or the like. The device may be constructed on a smaller scale than conventional surface plasmon resonance devices. The device generally includes a substrate, a radiation source, a radiation detector, and a reflector, among other elements.

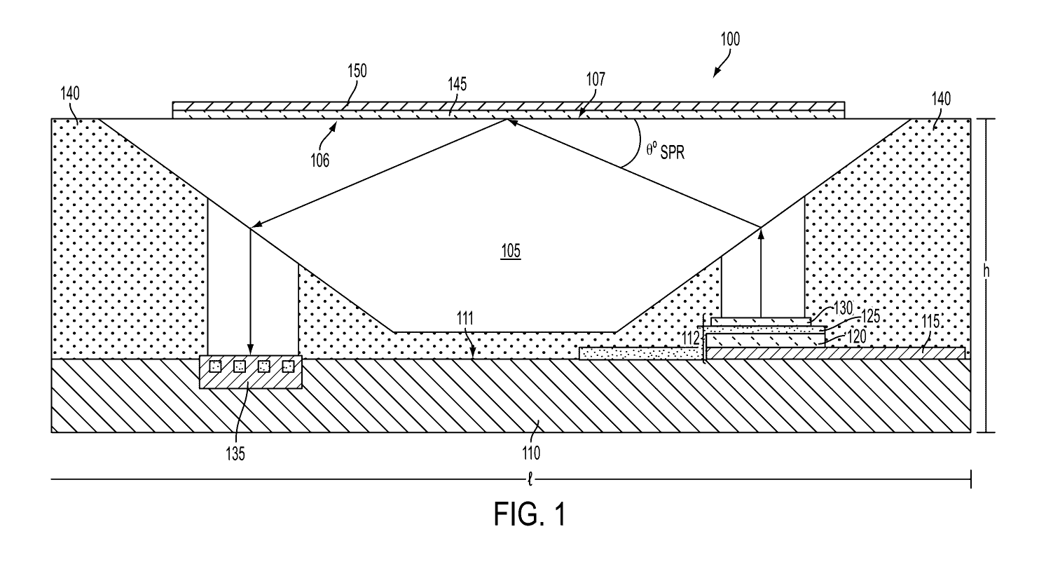

FIG. 1 depicts an illustrative biosensing device, generally designated 100, according to an embodiment. The biosensing device 100 may generally be configured to detect biofilm growth on a surface of a medical device, as described in greater detail herein. In some embodiments, the biosensing device 100 may be a microelectromechanical biosensing device. In some embodiments, the biosensing device 100 may be configured to communicate with an external device, such as a computing device and/or the like, to transmit data to the external device.

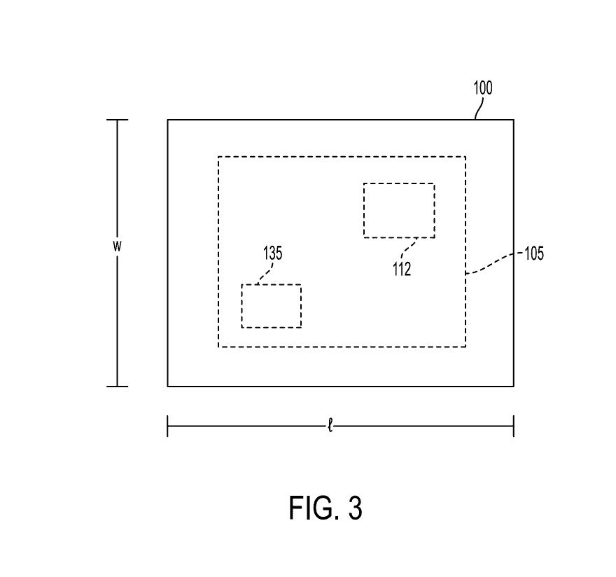

The biosensing device 100 may generally be sized and shaped such that it can be attached to a medical device. In some embodiments, the biosensing device 100 may be sized and shaped such that it can be integrated with a medical device. An illustrative sized biosensing device 100 may be a MEMS-scale device. Thus, the biosensing device 100 may have a length/of about 0.01 mm to about 2 mm. For example, the length/may be about 0.01 mm, about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, or any value or range between any two of these values (including endpoints). Similarly, as shown in FIG. 3, the biosensing device 100 may have a width w of about 0.01 mm to about 2 mm. For example, the width w may be about 0.01 mm, about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, or any value or range between any two of these values (including endpoints). In addition, as shown in FIG. 1, the biosensing device 100 may have a height h of about 0.01 mm to about 1 mm. For example, the height h may be about 0.01 mm, about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, or any value or range between any two of these values (including endpoints). In a particular embodiment, the biosensing device 100 may have a width w of about 1.0 mm, a length/of about 1.0 mm, and a height h of about 0.5 mm.

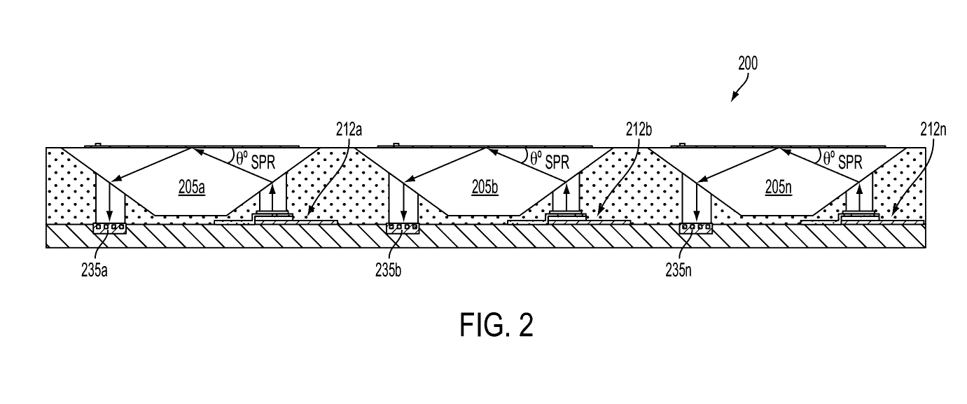

The biosensing device 100 may generally include a substrate 110, at least one radiation source 112 on the substrate, at least one radiation detector 135 on the substrate, and at least one reflector 105. The biosensing device 100 may include any number of radiation sources 112, radiation detectors 135, and reflectors 105. For example, as shown in FIG. 2, the biosensing device 200 may be an array of a plurality of radiation sources 212a . . . n, an array of reflectors 205a . . . n, and/or an array of radiation detectors 235a . . . n. For purposes of simplicity, as described herein, particularly with respect to FIG. 1, the biosensing device 100 includes one radiation source 112, one radiation detector 135, and one reflector 105.

The substrate 110 may generally be any substrate, particularly substrates suitable for supporting the various elements described herein. The substrate 110 may be silicon-based. An illustrative silicon-based substrate may include, but is not limited to, a silicon wafer. In some embodiments, the substrate 110 may be a portion of a medical device, as described in greater detail herein. In some embodiments, the substrate may have a plurality of surfaces. In such embodiments, the radiation source 112, the reflector 105, and the radiation detector 135 may all be arranged on a first surface 111 of the substrate.

The radiation source 112 may generally be a device that is configured to emit radiation. For example, the radiation source 112 may be an organic light emitting diode (OLED) excitation source. Thus, the OLED may be configured to produce single pixel light that spans a wide range of wavelengths. In some embodiments, the radiation source may be configured to emit radiation capable of inducing surface plasmon polariton excitation of sensor material 145, as described in greater detail herein. In various embodiments, the radiation source 112 may incorporate various materials that allow a wavelength of an emitted light to be continuously adjusted for a particular optical system. Such adjustments may be completed for adjustable variables that allow the generation of the optical path and/or achieve a surface plasmon polariton at the sensor material 145, as described in greater detail herein. The adjustable variables may include, for example, an emission wavelength of the radiation source 112, an index of refraction of the reflector 105, a bevel angle of the reflector, a complex index and thickness of the sensor material 145, and a complex index and thickness of a functionalization layer 150.

In a general form, the radiation source 112 may include a cathode 115 such as a metal cathode, an emitter 120 such as an OLED emitter on at least a portion of the cathode, a transparent anode 125 on at least a portion of the emitter, and a polarizer 130 on at least a portion of the transparent anode. The polarizer 130 may generally be configured to selectively transmit light through from the emitter 120 and/or other portions of the radiation source 112. The light may generally be polarized in a transverse electric field mode relative to a surface of the reflector 105.

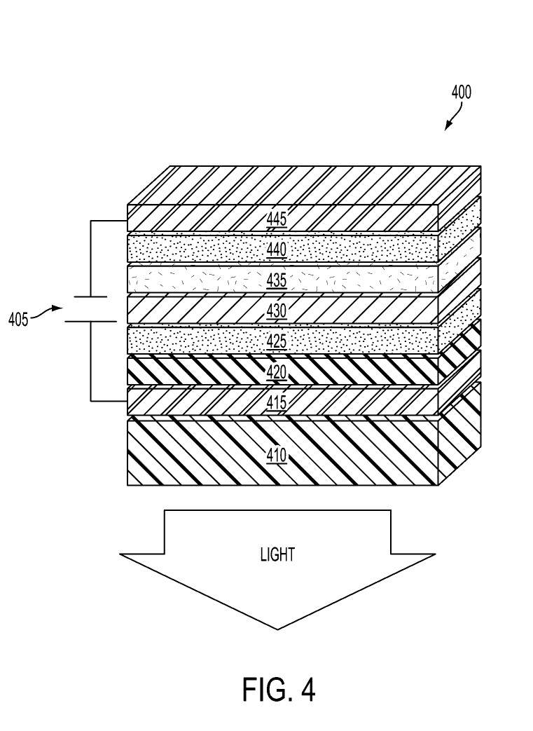

FIG. 4 depicts a side view of an illustrative radiation source according to an embodiment. As shown in FIG. 4, the radiation source 400 may include a transparent substrate 410, a transparent anode 415 on at least a portion of the transparent substrate, a hole injection layer 420 on at least a portion of the transparent anode, a hole transport layer 425 on at least a portion of the hole injection layer, an emissive layer 430 on at least a portion of the hole transport layer, a hole blocking layer 435 on at least a portion of the emissive layer, an electron transport layer 440 on at least a portion of the hole blocking layer, and a cathode 445 on at least a portion of the electron transport layer. A power input 405 may be connected to the transparent anode 415 and the cathode 445.

The transparent substrate 410 may generally be any substrate that allows passage of radiation as described herein. Illustrative transparent substrates 410 may include silicon-based substrates, mineral glass, transparent polymer substrates that support deposition of the transparent anode 415 (such as, for example, a polycarbonate substrate, a polystyrene substrate, and a polyacrylate substrate) and/or the like.

The transparent anode 415 and/or the cathode 445 may generally be transparent to the various wavelengths of radiation emitted from the emissive layer 430 such that radiation may be emitted from the radiation source 400. In some embodiments, the transparent anode 415 and/or the cathode 445 may be transparent to ambient light such that ambient light may pass through the radiation source 400. When a positive bias is applied across the transparent anode 415, the anode may inject holes (positive charge carriers) into the emissive layer 430. The holes may combine with electrons from the cathode 445 in the emissive layer 430 and generate excitons that cause luminescence. Accordingly, the emissive layer 430 may emit radiation, such as, for example, in the form of visible light. The radiation emitted from the emissive layer 430 may have a wavelength and a chirality. For example, the radiation may be circularly polarized and may have a left-handed or a right-handed orientation. Similarly, the radiation may be elliptically polarized and may have a left-handed or a right-handed orientation. In some embodiments, the transparent anode 415 and/or the cathode 445 may include an optically active reflective layer having morphologically stable glass-forming chiral nematic liquid crystals (GLCs). The optically active reflective layer may also include a cholesteric liquid crystal.

The hole injection layer 420 may generally be a layer for supplying holes (positive charge carriers) from the transparent anode 415 to the emissive layer 430. The hole injection layer 420 may include a mixture of metal fluoride and an organic compound, which is a material conventionally used to form a hole injection layer. The metal in the metal fluoride may be a Group 1 element or a Group 2 element, as non-limiting examples. As specific, non-limiting examples, the metal fluoride may be LiF, MgF₂, BaF, CsF, NaF, CaF₂, or the like, and may be prepared using various methods known to one of ordinary skill in the art. As non-limiting examples, the mixing molar ratio of the metal fluoride to the organic compound for forming the hole injection layer 420 may be about 1:1 to about 3:1. When the mixing molar ratio of the metal fluoride to the organic compound for forming the hole injection layer 420 is less than 1:1, the effect of a reduction in driving voltage may be insignificant, and the interface resistance according to time may increase. Conversely, when the mixing molar ratio of the metal fluoride to the organic compound is greater than 3:1, the driving voltage may increase.

The hole transport layer 425 may generally be configured to transport the holes from the hole injection layer 420. The hole transport layer 425 may include organic materials and inorganic materials. An illustrative example of an organic material may include an organic chromophore. The organic chromophore may include a phenyl amine, such as, for example, N,N-diphenyl-N,N-bis(3-methylphenyl)-(1,r-biphenyl)-4,4′-diamine (TPD). Other materials in the hole transport layer 425 may include (N,N-bis(3-methylphenyl)-N,N-bis(phenyl)-spiro (spiro-TPD), 4-4′-N,N′-dicarbazolyl-biphenyl (CBP), 4,4-, bis[N-(1-naphthyl)-N-phenylamino]biphenyl (NPD), a polyaniline, a polypyrrole, a poly(phenylene vinylene), copper phthalocyanine, an aromatic tertiary amine or polynuclear aromatic tertiary amine, a 4,4′-bis(p-carbazolyl)-1,1′-biphenyl compound, N,N,N′,N′-tetraarylbenzidine, poly(3,4-ethylenedioxythiophene) (PEDOT)/polystyrene para-sulfonate (PSS) derivatives, poly-N-vinylcarbazole derivatives, polyphenylenevinylene derivatives, polyparaphenylene derivatives, polymefhacrylate derivatives, poly(9,9-octylfluorene) derivatives, poly(spiro-fluorene) derivatives, N,N′-di(naphthalene-1-yl)-N,N′-diphenyl-benzidine (NPB), tris(3-methylphenylphenylamino)-triphenylamine (m-MTDATA), poly(9,9′-dioctylfluorene-co-N-(4-butylphenyl)diphenylamine (TFB), and 2,2′7,7′-tetrakis[N-naphthalenyl(phenyl)-amino]-9,9-spirobifluorene (spiro-2NPB). Illustrative examples of inorganic materials may include, for example, inorganic semiconductor materials capable of transporting holes. The inorganic material may be amorphous or polycrystalline. Hole transport materials having, for example, an inorganic material such as an inorganic semiconductor material, may be deposited at a low temperature, for example, by a known method, such as a vacuum vapor deposition method, an ion plating method, sputtering, inkjet printing, sol-gel, and/or the like. The various materials of the hole transport layer 425 may be deposited by known methods, such as, for example, a vacuum vapor deposition method, a sputtering method, a dip-coating method, a spin-coating method, a casting method, a bar-coating method, a roll-coating method, and the like. In particular embodiments, organic layers may be deposited under ultra-high vacuum (for example, less than or equal to about 10⁻⁸ torr), high vacuum (for example, about 10⁻⁸ torr to about 10⁻⁵ torr), or low vacuum conditions (for example, about 10⁻⁵ torr to about 10⁻³ torr). In some embodiments, the hole-injection layer 420 may be combined with the hole transport layer 425 that has been doped, such as, for example, p-type doped.

In some embodiments, the emissive layer 430 may include small molecule emissive materials. Illustrative small molecule emission materials may include, but are not limited to, tris(8-hydroxyquinolinato)aluminum (Alq3), triphenylamine, perylene, rubene, quinacridone, any derivative thereof, and any combination thereof. The small molecule emissive materials may have an emission spectrum of about 500 nm.

In some embodiments, the emissive layer 430 may include an emissive polymer. Illustrative emissive polymers may include, but are not limited to, polyparaphenylene-vinylene, poly(p-phenylene vinylene), poly(naphthalene vinylene), polyfluorene, and any combination thereof. The emissive polymer may have an emission spectrum of about 500 nm.

The hole blocking layer 435 may generally be used to prevent carrier escape from the radiation source 400. The hole blocking layer 435 may be n-doped sufficiently such that the hole blocking layer does not block electron injection into the emissive layer 430. Accordingly, an illustrative hole blocking layer may include a nominally Al_{0.2}Ga_{0.8}N layer heavily doped with Si to about 3×10¹⁹ cm⁻³. Without the hole blocking layer 435, electroluminescence (EL) output power in the radiation source 400 with a partially relaxed lower InGaN waveguide layer may be reduced by as much as about 50% relative to a reference light-emitting diode (LED). With the hole blocking layer 435, the output power for the radiation source 400 on a relaxed InGaN layer may be similar to that of the reference LED.

The electron transport layer 440 may generally be configured to transport electrons from the cathode 445 to the emissive layer 430. The electron transport layer 440 may include, for example, 3-(4-biphenyl)-4-phenyl-5-tert-butylphenyl-1,2,4-triazole, 4-(naphthalen-1-yl)-3,5-diphenyl-4H-1,2,4-triazole, 2,9-bis(naphthalen-2-yl)-4,7-diphenyl-1,10-phenanthroline, 2,7-bis[2-(2,2′-bipyridine-6-yl)-1,3,4-oxadiazo-5-yl]-9,9-dimethylfluorene, 1,3-bis[2-(4-tert-butylphenyl)-1,3,4-oxadiazo-5-yl]benzene, Tris(2,4,6-trimethyl-3-(pyridin-3-yl)phenyl)borane, 1-methyl-2-(4-(naphthalen-2-yl)phenyl)-1H-imidazo[4,5f] [1,10] phenanthroline, 2-(naphthalen-2-yl)-4,7-diphenyl-1,10-phenanthroline, 2,7-bis[2-(2,2′-bipyridine-6-yl)-1,3,4-oxadiazo-5-yl]-9,9-dimethylfluorene, 1,3-bis[2-(4-tert-butylphenyl)-1,3,4-oxadiazo-5-yl]benzene, Tris(2,4,6-trimethyl-3-(pyridin-3-yl)phenyl)borane, 1-methyl-2-(4-(naphthalen-2-yl)phenyl)-1H-imidazo[4,5f][1,10] phenanthroline, 2-(naphthalen-2-yl)-4,7-diphenyl-1,10-phenanthroline, Phenyl-dipyrenylphosphineoxide, 3,3′5,5′-tetra[(m-pyridyl)-phen-3-yl]biphenyl, 1,3,5-tri[(3-pyridyl)-phen-3-yl]benzene, 4,4′-bis(4,6-diphenyl-1,3,5-triazin-2-yl)biphenyl, 1,3-bis[3,5-di(pyridin-3-yl)phenyl]benzene, Diphenylbis(4-(pyridin-3-yl)phenyl)silane, 3,5-di(pyren-1-yl)pyridine, 1,3,5-tri(p-pyrid-3-yl -phenyl)benzene, 2,4,6-tris(3′-(pyridin-3-yl)biphenyl-3-yl)-1,3,5-triazine, 4,6-bis(3,5-di(pyridin-3-yl)phenyl)-2-methylpyrimidine, Poly[(9,9-bis(3′-((N,N-dimethyl)-N-ethyl ammonium)-propyl)-2,7-fluorene)-alt-2,7-(9,9-dioctylfluorene)], 1,3,5-tris(4-(pyridin-4-yl)quinolin-2-yl)benzene, 2-(biphenyl-4-yl)-4,6-bis(4′-(pyridin-2-yl)biphenyl-4-yl)-1,3,5-triazine, and 2,4-di(biphenyl-4-yl)-6-(4′-(pyridin-2-yl)biphenyl-4-yl)-1,3,5-triazine.

In some embodiments, the cathode 445 may be the closest layer to the substrate 110 (FIG. 1). The various layers 410-445 may generally be deposited by any method known in the art, such as patterning, sputtering, sublimating, casting, and/or the like. For example, a metal contact may be formed on the substrate 110 (FIG. 1) to form the cathode layer 445. The emissive layer 430 may be patterned into individual pixels and the transparent anode 415 may be patterned thereon. The various other layers may also be patterned thereon as appropriate.

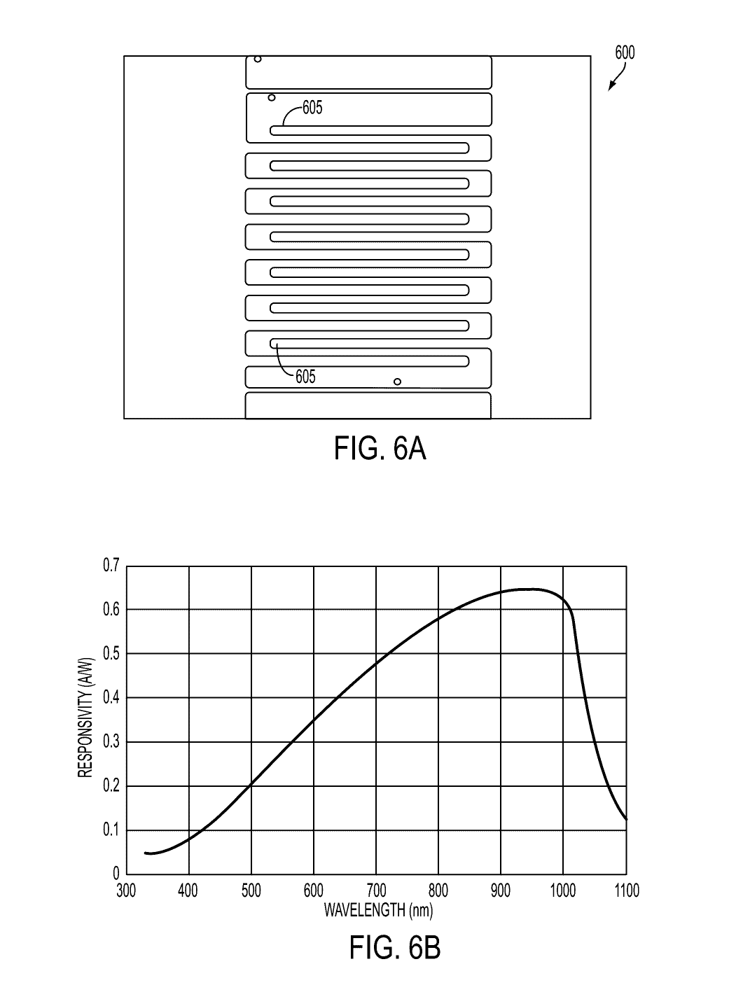

Referring back to FIG. 1, the radiation detector 135 may generally be configured to detect at least the radiation emitted by the radiation source 112. In particular embodiments, the radiation detector 135 may be configured to detect an intensity of the radiation reflected by the reflector 105, as described in greater detail herein. Thus, any radiation detector now known or later developed may be used, particularly radiation detectors that are suitable for the various purposes described herein. For example, the radiation detector 135 may include any type of active or passive radiation-sensitive material, which may generally be contained or monitored for indication of interaction with radiation. The radiation detector 135 may be a composite of materials, such as scintillator materials in combination with a photo-multiplier tube (PMT). An illustrative radiation detector 135 may be a photodetector, particularly photodetectors that are commonly known in the art. The radiation detector 135 may include a silicon photodiode junction detector formed on a silicon wafer substrate with a p-n junction. FIG. 6A depicts an illustrative radiation detector 135 according to an embodiment. As shown in FIG. 6A, the radiation detector 600 may have an interdigitated well structure. Metallization contacts 605 may be patterned onto p and n dopant wells for external contact. Such radiation detectors depicted in FIG. 6A are commonly known in the art and have a response curve that trends towards a higher sensitivity at near-infrared wavelengths (about 1 μm) of the electromagnetic spectrum, as depicted in FIG. 6B.

Referring again to FIG. 1, the reflector 105 may generally be arranged on the substrate 110 such that the radiation emitted from the radiation source 112 is reflected toward the radiation detector 135. Accordingly, as depicted by the black arrows, the radiation is reflected one or more times from the radiation source 112 to the radiation detector 135. Thus, the reflector 105 may generally be any optical device that has reflective properties, such as a reflecting surface that reflects electromagnetic radiation. Illustrative examples of a reflector may include one or more mirrors of various shape and form, including, but not limited to, flat mirrors and/or mirrors formed with spherical or cylindrical surfaces. A mirror may also include objects with reflective surfaces that have somewhat less reflective efficiency than a conventional mirror, such as a thin aluminum sheet with a protection layer on its top. In some embodiments, the reflector 105 may use internal reflection, such as a prism and/or other devices that include one or more substantially transparent or translucent components. In particular embodiments, the reflector 105 may be a metallized prism.

As used herein, a reflector 105 is considered to be “one” reflector if the geometric form of its light reflecting face can be described by a simple mathematical equation. If the geometric form of the light reflecting face of a reflector 105 can only be described by two or more simple mathematical equations, the reflector is considered to include two or more reflectors. For example, if a segment of the light reflecting face of a reflector 105 can be expressed by a linear function while the other segment of the light reflecting face of the reflector can be expressed by that of a cylindrical trough, this reflector may be considered to include two reflectors, even if the light reflecting surface of the reflector body is a smooth compound surface. In particular embodiments, the reflector 105 may be a “perfect” reflector that has no loss of light and a reflecting surface that precisely follows a desired mathematical description or an ideal design.

In some embodiments, the reflector 105 may include sensor material 145 on at least a portion thereof. In particular embodiments, the sensor material 145 may be a film of sensor material. The sensor material 145 may generally be disposed on a surface of the reflector 105 that is opposite to a surface where the radiation is reflected. Thus, as shown in FIG. 1, a first surface 106 of the reflector 105 may reflect radiation, and the opposite surface 107 may contain the sensor material 145. The sensor material 145 may generally be a noble metal thin film that can be excited via surface plasmon polaritons, as described in greater detail herein. Illustrative noble metals that may be used in the sensor material 145 may include, but are not limited to, gold, silver, rhodium, ruthenium, palladium, osmium, iridium, platinum, and combinations thereof. One or more other metals that resist corrosion but are not considered “noble” metals may also be incorporated in the sensor material 145. Other illustrative metals include titanium, niobium, and tantalum.

In some embodiments, the sensor material 145 may be deposited on the reflector 105 such that it has a desired thickness. The thickness of the sensor material 145 may be about 1 nm to about 100 nm. For example, the sensor material 145 may have a thickness of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, or any value or range between any two of these values (including endpoints).

In various embodiments, a functionalization layer 150 may be deposited on at least a portion of the sensor material 145. The functionalization layer 150 may generally be configured to mimic a surface of a medical device, particularly medical devices upon which the biosensing device 100 is deposited and/or integrated. This may be achieved by presenting the same or a substantially similar polymer and/or organic chemical surface as that of the medical device to mimic its reactivity. If the medical device surface is a polymer such as polyethylene, the chemical functions may be linear alkanes, and the surface of the biosensing device 100 may be functionalized with the functionalization layer 150 with long-chain (about C-10 to about C-20) alkane thiols and/or ester functionalized thiols. If the medical device surface is a hydrogel, then the functionalization layer may include oligoethylene glycol thiols. In such a configuration, the thiols may be attached to oligo groups that are identical to monomers of the various polymeric materials used to form the biosensing device 100. For example, if the medical device surface is polytetrafluoroethylene (PTFE), the functionalization layer 150 may include CF₂ groups on the thiols. Similarly, if the medical device surface is an acrylic-based material, the functionalization layer 150 may include acrylate esters on the thiol. In addition, if the medical device surface incorporates polyethylene terephthalate (PET), the functionalization layer 150 may include terephthalate esters with the thiol.

To ensure that the various components of the biosensing device 100 are appropriately aligned on the substrate 110, one or more support devices 140 may be used. The support device 140 may generally be any device configured to stabilize one or more components on the substrate 110. For example, the support device 140 may be configured to stabilize the reflector 105 against the substrate 110. In some embodiments, the support device 140 may ensure that the reflector 105 provides a clear optical path from the radiation source 112 to the radiation detector 135. An illustrative support device 140 may be, but is not limited to, a support slab. In some embodiments, the support slab may be, for example, a rigid polymer material, a glass material, a ceramic material, and/or the like.

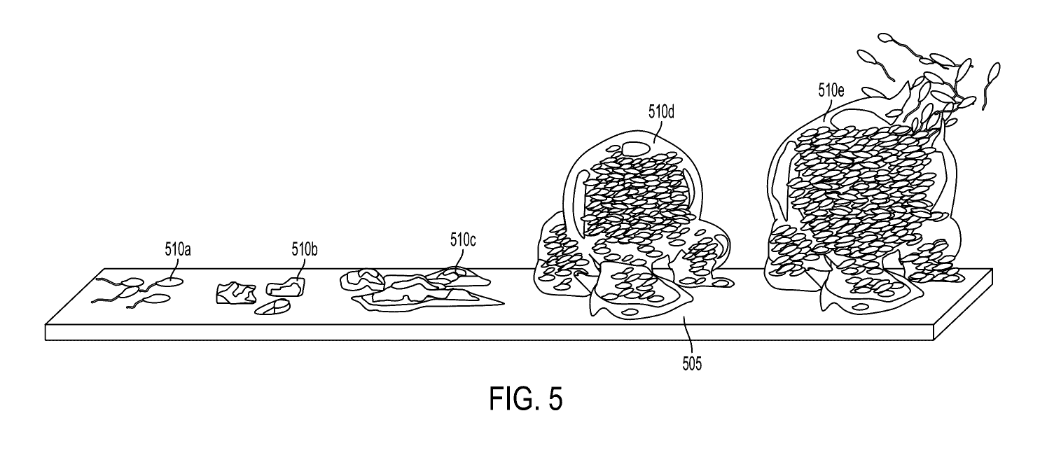

As depicted in FIG. 5, a biofilm 510a-e may grow on a portion of the reflector 505. A maturity of the biofilm 510a-e growth may cause a change in the intensity of the light reflected by the reflector 505, as described herein. The five stages of biofilm development may include an initial attachment 510a, an irreversible attachment 510b, a first maturation 510c, a second maturation 510d, and a dispersion 510d.

Referring also to FIG. 1, the biosensing device 100 may generally function by emitting, via the radiation source 112, electromagnetic radiation that is reflected by the reflector 105 toward the radiation detector 135. As previously described herein, the radiation detector 135 may be configured to detect an intensity of the radiation. When a biofilm growth is present on at least a portion of the reflector 105, it may cause a change in the intensity of the radiation relative to an intensity when no biofilm is present. The different intensity may also be detected by the radiation detector 135. More particularly, the emitted radiation that reflects off the reflector 105 is directed to the radiation detector 135. According to the theory of SPR bio-detection, the angular dependence of the reflectivity is a function of the concentration of biomolecules bound at the surface of the functionalization layer 150. The specularly reflected radiation intensity (which is indicative of the biofilm bound on the sensor) is directed to and monitored by the radiation detector 135. The angle at which the p-polarized light may resonate a surface plasmon polariton of the metal in the sensor material 145 (capable of detecting a binding induced index change) is dependent on the thickness and complete index of three phases: (1) the sensor material (constant properties and index); (2) the functionalization layer 150 (constant properties); and (3) the biofilm (thickness and properties are a function of environment and time).

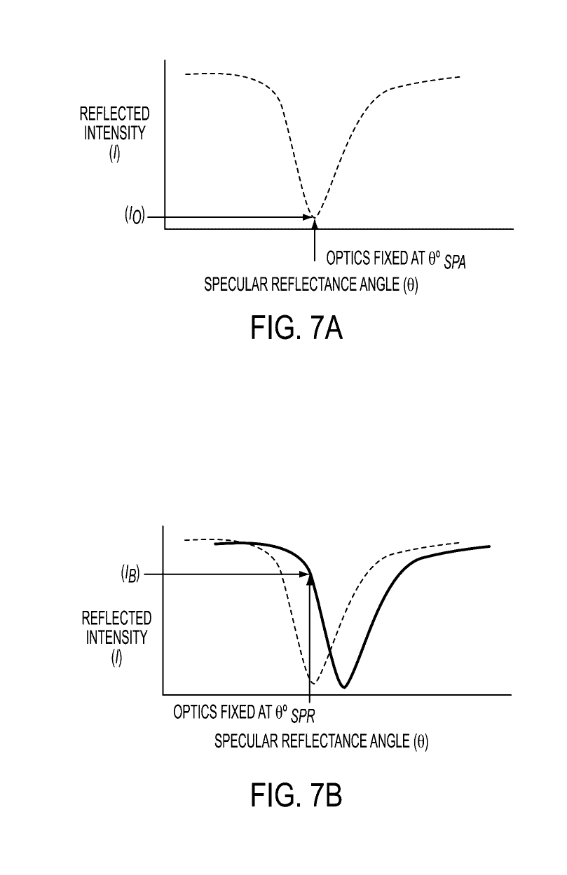

For a given wavelength and molecular binding state, the variation of a specular reflectance angle θ°_{SPR} versus the intensity may reveal the resonance absorption minimum, which is indicative of the surface plasmon polariton as shown in the graph in FIG. 7A. Assuming the optical intensity as measured by the radiation detector 135 is obtained when a surface of the biosensing device 100 is clean (for example, no biofilm growth), a minimum intensity Iₒ is detected when the biosensing device is configured to be fixed at the specular reflectance angle θ°_{SPR}. As the biofilm binds and grows (in thickness and/or in density) on the biosensing device 100, the optical state is transformed. The addition of the biofilm near the functionalization layer 150 may change the local index of refraction and the transmission of the specular reflectance evanescent wave. The net effect is a shift in the intensity I versus the angle θ as shown in the graph in FIG. 7B. As shown in the graph, the reflected intensity I changes, but the optical geometry is fixed at the original specular reflectance angle θ°_{SPR}. Thus, the radiation detector 135 may measure a higher specular optical intensity since the system has a different characteristic curve.

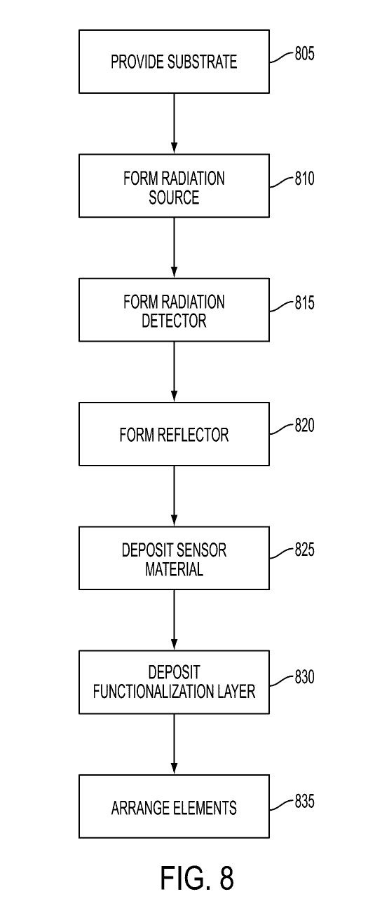

FIG. 8 depicts a flow diagram of an illustrative method of forming a biosensing device, such as the biosensing device described herein, according to an embodiment. The method may include providing 805 a substrate, forming 810 a radiation source on the substrate, forming 815 a radiation detector on the substrate, and forming 820 a reflector on the substrate. As previously described herein, the various forming 810-820 processes may generally be completed by any method of deposition. In some embodiments, forming 820 the reflector on the substrate may include depositing a support slab on the substrate and molding the support slab such that the support slab is configured to be filled with a reflector material. Accordingly, the reflector material may be placed in the support slab. For example, a liquid or semisolid reflector material may be placed in the support slab, where it may be allowed to harden.

In various embodiments, the method may further include depositing 825 sensor material on at least a portion of the reflector. As previously described herein, the sensor material may be a noble metal material disposed on a surface of the reflector that is opposite to a surface where the radiation is reflected. The sensor material may generally be deposited 825 at a uniform thickness, such as a thickness of about 1 nm to about 100 nm.

In various embodiments, the method may also include depositing 830 a functionalization layer on at least a portion of the film sensing material. As previously described herein, the functionalization layer may be configured to mimic a surface of a medical device. The functionalization layer may be deposited 830 such that it includes a long chain alkane thiol and/or an ester functionalized thiol to mimic the surface of the medical device, as previously described herein.

In various embodiments, the method may also include arranging 835 the various elements. For example, in some embodiments, the radiation source, the radiation detector, and the reflector may be arranged 835 such that the light reflected from the radiation source is reflected by the reflector towards the radiation detector, as described in greater detail herein. In some embodiments, arranging 835 may be completed to place at least one radiation source and at least one radiation detector in an array configuration. Arranging 835 may also include placing a plurality of prisms in an array configuration.

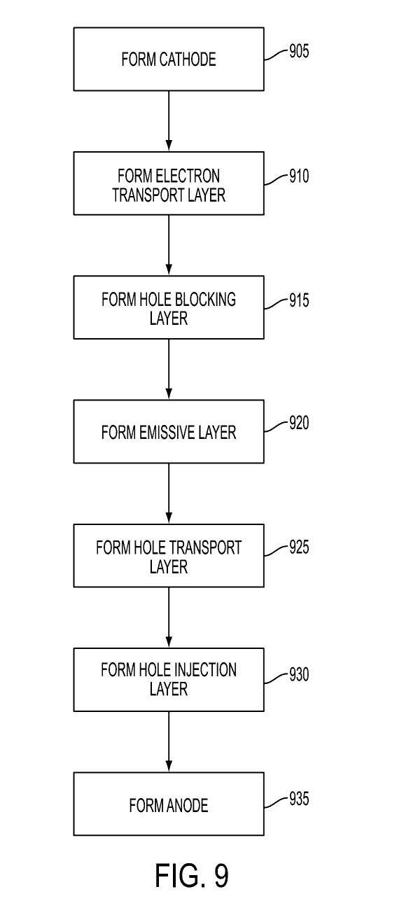

Forming 810 the radiation source may include a plurality of additional processes, as described herein with respect to FIG. 9. The method may include forming 905 a cathode on the substrate, forming 910 an electron transport layer on at least a portion of the cathode, forming 915 a hole blocking layer on at least a portion of the electron transport layer, forming 920 an emissive layer on at least a portion of the hole blocking layer, forming 925 a hole transport layer on at least a portion of the emissive layer, forming 930 a hole injection layer on at least a portion of the hole transport layer, and forming 935 a transparent anode on at least a portion of the hole injection layer. As previously described herein, the various forming 905-935 processes may include any method of deposition. In some embodiments, forming 935 the transparent anode may include depositing indium tin oxide on at least a portion of the hole injection layer.

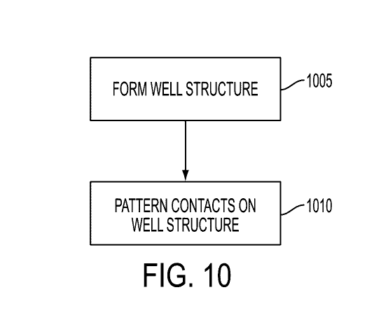

Forming 815 the radiation detector may include a plurality of additional processes, as described herein with respect to FIG. 10. The method may include forming 1005 a well structure and patterning 1010 a plurality of metallization contacts on the well structure. In some embodiments, the well structure may have a p dopant well and an n dopant well, and the metallization contacts may be patterned 1010 in an interdigitated manner on the p dopant well and on the n dopant well such that a resulting radiation detector, as depicted in FIG. 6A, is formed.

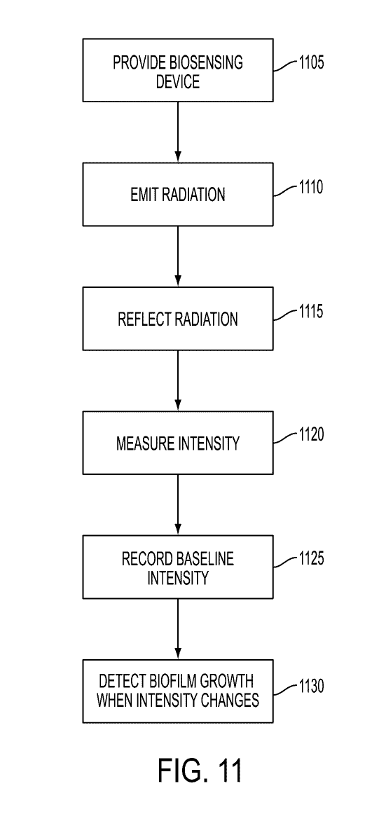

FIG. 11 depicts a flow diagram of an illustrative method of detecting a presence of a biofilm on a surface according to an embodiment. The method may include providing 1105 a biosensing device, such as, for example, one of the biosensing devices described herein. The method may further include emitting 1110 radiation, such as from the radiation source, toward the reflector at an angle. The emitted radiation may be reflected 1115 by the reflector. The radiation reflected 1115 by the reflector may be a specular reflection of the radiation incident on the reflector, as described in greater detail herein. The intensity of the reflected 1115 radiation may be measured 1120, such as with the radiation detector, as described herein. A baseline intensity value based on the intensity of the reflected 1115 radiation may be recorded 1125. When a biofilm grows on the biosensing device as described herein, it may cause a change in the intensity of the reflected radiation with respect to the recorded 1125 baseline value. Such a change may be detected 1130 by the radiation detector, as described in greater detail herein. In some embodiments, the change may be an increase in intensity with respect to the recorded 1125 baseline value. In some embodiments, the biofilm may be detected when a growth of the biofilm on the film of sensor material causes a change in the local index of refraction of the reflector, which may be measured by the radiation detector, as described in greater detail herein.

### EXAMPLES

### Example 1

### MEMS Biosensing Device

A MEMS biosensing device that is configured to be integrated into a surface of an artificial hip will be used to sense growth of biofilms on the artificial hip after it is implanted in a patient. When the MEMS biosensing device senses the biofilm growth, it will be configured to transmit data to a computing device, which is used to alert medical personnel of a possible infection in the patient so that the infection can be treated before it causes serious illness.

The MEMS biosensing device will have a length of about 1.0 mm, a width of about 1.0 mm, and a height of about 0.5 mm. A device of such size will be small enough to be implanted in the artificial hip without hindering any of the hip's function when implanted into a patient.

The MEMS biosensing device will include a silicon substrate having 10 radiation sources, 10 radiation detectors, and one reflector arranged thereon. Each radiation source is a OLED configured to emit light. The OLED will be a conventional OLED having a stack of components, including (in order from the substrate) a cathode, an electron transport layer, a hole blocking layer, an emissive layer, a hole transport layer, a hole injection layer, a transparent anode, and a transparent substrate. The emissive layer will include Alq3 and will have an emission spectrum of about 550 nm. Each radiation detector will be a silicon photodiode junction detector configured to measure the intensity of the light emitted by an OLED. The reflector will be an inverted trapezoidal shaped prism. The prism will be coated with a 50 nm thick noble metal thin film sensor made of gold. A functionalization layer will be placed over the noble metal thin film sensor to mimic the properties of the ultra-high molecular weight polyethylene composition of the hip implant.

The various components will be arranged on the substrate such that each OLED is paired with a corresponding detector. Accordingly, each OLED will emit light that is reflected through the prism and detected only by its corresponding detector. The light reflected by the OLED will reflect off a surface of the prism that is opposite the noble metal thin film sensor.

### Example 2

### Detecting Biofilm Growth

The MEMS biosensing device described above with respect to Example 1 will generally detect any biofilm growth on the surface of the artificial hip. Such biofilm growth is unwanted once the artificial hip is implanted in a patient because the biofilm is indicative of an infection. However, the MEMS biosensing device will be configured to detect very small traces of biofilms immediately after the initial attachment phase. Accordingly, the detection can be used to notify medical personnel of the growth, which can be treated before a bigger issue results that is deleterious to the health of the patient.

The MEMS biosensing device will function by directing each OLED to emit light. The light will be reflected by the prism and detected by the corresponding detector. Once the hip implant has been placed in the patient, this will be completed to establish a baseline intensity of the light emitted from each OLED. Each OLED will be configured to emit light every 10 minutes. As described herein, the emitted light will increase in intensity when a biofilm presence is detected on the functionalization layer surface. This increase in intensity will immediately be sensed by the sensor, which will send a signal to an external computing device. The external computing device will notify medical personnel to take appropriate action.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera As a non-limiting example, each range discussed herein can be readily broken down into a lower third, middle third and upper third, et cetera As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A biosensing device comprising:
a substrate;
at least one radiation source on the substrate;
at least one radiation detector on the substrate; and
at least one reflector arranged on the substrate such that radiation emitted from the at least one radiation source is reflected toward the at least one radiation detector, and the at least one radiation detector being configured to detect an intensity of the radiation reflected from the at least one reflector,
wherein a biofilm growth on a portion of the at least one reflector causes a change in the intensity of the radiation reflected from the at least one reflector relative to radiation reflected from the reflector in the absence of the biofilm growth.

2. The biosensing device of claim 1, wherein the at least one radiation source, the at least one reflector, and the at least one radiation detector are arranged on a same side of the substrate.

3. The biosensing device of claim 1, wherein the at least one reflector comprises a prism.

4. The biosensing device of claim 1, wherein the at least one radiation source and the at least one radiation detector are arranged in an array configuration, and the at least one reflector comprises a plurality of prisms arranged in the array configuration.

5. The biosensing device of claim 1, further comprising at least one support device configured to stabilize the at least one reflector against the substrate.

6. The biosensing device of claim 1, further comprising a film of sensor material on at least a portion of the reflector, the sensor material being disposed on a surface of the reflector opposite to a surface where the radiation is reflected.

7. The biosensing device of claim 6, wherein the sensor material comprises at least one of gold, silver, and rhodium.

8. The biosensing device of claim 6, wherein the film of sensor material has a thickness of about 1 nm to about 100 nm.

9. The biosensing device of claim 6, further comprising a functionalization layer on at least a portion of the film of sensor material, wherein the functionalization layer is configured to mimic a surface of a medical device.

10. The biosensing device of claim 9, wherein the functionalization layer comprises at least one of a long chain alkane thiol or an ester functionalized thiol.

11. The biosensing device of claim 6, wherein the at least one radiation source is configured to emit radiation capable of inducing surface plasmon polariton excitation of the sensor material.

12. The biosensing device of claim 1, wherein the at least one radiation source comprises:
a metal cathode;
an organic light emitting diode emitter on at least a portion of the metal cathode;
a transparent anode on at least a portion of the organic light emitting diode emitter; and
a polarizer on at least a portion of the transparent anode, the polarizer configured to selectively transmit light therethrough from the organic light emitting diode emitter, wherein the light is polarized in a transverse electric field mode relative to a surface of the at least one reflector.

13. The biosensing device of claim 1, wherein the radiation source comprises an organic light emitting diode.

14. The biosensing device of claim 13, wherein the organic light emitting diode comprises at least one small molecule emission material selected from tris(8-hydroxyquinolinato)aluminum, triphenylamine, perylene, rubene, quinacridone, any derivative thereof, or any combination thereof.

15. The biosensing device of claim 13, wherein the organic light emitting diode comprises at least one emissive polymer selected from polyparaphenylene-vinylene, poly(p-phenylene vinylene), poly(naphthalene vinylene), polyfluorene, and any combination thereof.

16. The biosensing device of claim 1, wherein the at least one radiation source comprises a transparent substrate, a transparent anode on at least a portion of the transparent substrate, a hole injection layer on at least a portion of the transparent anode, a hole transport layer on at least a portion of the hole injection layer, an emissive layer on at least a portion of the hole transport layer, a hole blocking layer on at least a portion of the emissive layer, an electron transport layer on at least a portion of the hole blocking layer, and a cathode on at least a portion of the electron transport layer.

17. The biosensing device of claim 1, wherein the substrate is a silicon wafer.

18. The biosensing device of claim 1, wherein the radiation detector is a p-n junction silicon photodetector.

19. The biosensing device of claim 1, wherein the biosensing device has a length of about 0.01 mm to about 2 mm.

20. The biosensing device of claim 1, wherein the biosensing device has a width of about 0.01 mm to about 2 mm.

21. The biosensing device of claim 1, wherein the biosensing device has a height of about 0.01 mm to about 1 mm.

22. The biosensing device of claim 1, wherein the biosensing device has a width of about 1 mm, a length of about 1 mm, and a height of about 0.5 mm.

23. The biosensing device of claim 1, wherein the biosensing device is configured to be attached to a medical device.

24. The biosensing device of claim 1, wherein the biosensing device is configured to be integrated with a medical device.

25. The biosensing device of claim 1, wherein the biosensing device is configured to detect biofilm growth on a surface of a medical device.

26. The biosensing device of claim 1, wherein the biosensing device is a microelectromechanical biosensing device.

27. A method of forming a biosensing device, the method comprising:
providing a substrate;
forming at least one radiation source on the substrate;
forming at least one radiation detector on the substrate; and
forming at least one reflector on the substrate, the at least one reflector being configured to reflect radiation emitted from the at least one radiation source toward the at least one radiation detector, and the at least one radiation detector being configured to detect an intensity of the radiation reflected from the at least one reflector.

28. The method of claim 27, wherein the at least one reflector comprises a prism.

29. The method of claim 27, further comprising arranging the at least one radiation source and the at least one radiation detector in an array configuration, wherein the at least one reflector comprises a plurality of prisms arranged in the array configuration.

30. The method of claim 27, wherein forming the at least one reflector comprises:
depositing a support slab on the substrate; and
molding the support slab such that the support slab is configured to be filled with a reflector material.

31. The method of claim 30, wherein the support slab comprises at least one of a rigid polymer material, a glass material, or a ceramic material.

32. The method of claim 27, further comprising depositing a film of sensor material on at least a portion of the reflector, the sensor material being disposed on a surface of the reflector opposite to a surface where the radiation is reflected.

33. The method of claim 32, wherein the film of sensor material comprises at least one of gold, silver, and rhodium.

34. The method of claim 32, wherein depositing the film of sensor material comprises depositing the film of sensor material at a thickness of about 1 nm to about 100 nm.

35. The method of claim 27, further comprising depositing a functionalization layer on at least a portion of the film of sensor material, wherein the functionalization layer is configured to mimic a surface of a medical device.

36. The method of claim 35, wherein the functionalization layer comprises at least one of a long chain alkane thiol or an ester functionalized thiol.

37. The method of claim 35, wherein the radiation source comprises an organic light emitting diode.

38. The method device of claim 37, wherein the organic light emitting diode comprises at least one small molecule emission material selected from tris(8-hydroxyquinolinato)aluminum, triphenylamine, perylene, rubene, quinacridone, any derivative thereof, and any combination thereof.

39. The method device of claim 37, wherein the organic light emitting diode comprises at least one emissive polymer selected from polyparaphenylene-vinylene, poly(p-phenylene vinylene), poly(naphthalene vinylene), polyfluorene, and any combination thereof.

40. The method of claim 27, wherein forming the radiation source comprises:
forming a cathode on the substrate;
forming an electron transport layer on at least a portion of the cathode;
forming a hole blocking layer on at least a portion of the electron transport layer;
forming an emissive layer on at least a portion of the hole blocking layer;
forming a hole transport layer on at least a portion of the emissive layer;
forming a hole injection layer on at least a portion of the hole transport layer; and
forming a transparent anode on at least a portion of the hole injection layer.

41. The method of claim 40, wherein forming the transparent anode comprises depositing indium tin oxide on the at least a portion of the hole transport layer.

42. The method of claim 27, wherein forming the radiation detector comprises
forming a well structure having a p dopant well and an n dopant well; and
patterning a plurality of metallization contacts in an interdigitated manner on the p dopant well and on the n dopant well.

43. A method of detecting a presence of a biofilm on a surface, the method comprising:
providing a biosensing device comprising:
a substrate;
at least one radiation source on the substrate;
at least one radiation detector on the substrate;
at least one reflector arranged on the substrate such that radiation emitted from the at least one radiation source is reflected toward the at least one radiation detector, the at least one radiation detector being configured to detect an intensity of the radiation reflected from the at least one reflector; and
a film of sensor material on a surface of the at least one reflector opposite to a surface where the radiation is reflected;
emitting radiation from the radiation source toward the reflector at an angle;
reflecting the radiation via the reflector toward the radiation detector, wherein the radiation reflected from the reflector is a specular reflection of the radiation incident on the reflector;
measuring an intensity of the radiation reflected from the reflector using the radiation detector;
recording a baseline intensity value based on the intensity of reflected radiation from radiation incident on the reflector at the angle; and
detecting a biofilm presence when a growth of the biofilm on the sensor material causes a change in the intensity of reflected radiation from radiation incident on the reflector at the angle, with respect to the recorded baseline intensity value.

44. The method of claim 43, further comprising detecting a biofilm presence when a growth of the biofilm on the film of sensor material causes a change in a local index of refraction of the reflector.

45. The method of claim 43, wherein the change in the intensity is an increase in intensity with respect to the recorded baseline intensity value.

46. The method of claim 43, wherein the reflector comprises a prism.

47. The method of claim 43, wherein the at least one radiation source and the at least one radiation detector are arranged in an array configuration, and the at least one reflector comprises a plurality of prisms arranged in the array configuration.

48. The method of claim 43, wherein the film of sensor material comprises at least one of gold, silver, and rhodium.

49. The method of claim 43, wherein the film of sensor material has a thickness of about 1 nm to about 100 nm.

50. The method of claim 43, wherein emitting radiation from the radiation source toward the reflector induces surface plasmon polariton excitation of the sensor material.

51. The method of claim 43, wherein the radiation excitation source comprises an organic light emitting diode.

52. The method of claim 51, wherein the organic light emitting diode comprises at least one small molecule emission material selected from tris(8-hydroxyquinolinato)aluminum, triphenylamine, perylene, rubene, quinacridone, any derivative thereof, and any combination thereof.

53. The method of claim 51, wherein the organic light emitting diode comprises at least one emissive polymer selected from polyparaphenylene-vinylene, poly(p-phenylene vinylene), poly(naphthalene vinylene), polyfluorene, and any combination thereof.

54. The method of claim 43, wherein the radiation detector is a p-n junction silicon photodetector.
